(19) United States
(12) Patent Application Publication
(10) Pub. No.: US 2022/0096449 A1
(43) Pub. Date: Mar. 31, 2022

(54) METHOD OF TREATMENT WITH TRADIPITANT

(71) Applicant: Vanda Pharmaceuticals Inc., Washington, DC (US)

(72) Inventors: Mihael H. Polymeropoulos, Potomac, MD (US); Gunther Birznieks, Chevy Chase, MD (US); Changfu Xiao, Vienna, VA (US)

(21) Appl. No.: 17/296,331

(22) PCT Filed: Dec. 3, 2019
(86) PCT No.: PCT/US2019/064246

§ 371 Date: May 24, 2021

Related U.S. Application Data
(60) Provisional application No. 62/774,840, filed on Dec. 3, 2018.

(51) Int. Cl. A61K 31/444

(52) U.S. Cl. CPC A61K 31/444

(57) ABSTRACT

Disclosed herein is a method of treatment of gastroparesis and symptoms thereof, comprising treatment with the NK-1 receptor antagonist, tradipitant.

### CROSS REFERENCE TO RELATED APPLICATIONS

The present application claims the benefit of U.S. provisional application No. 62/774,840, filed Dec. 3, 2018.

### BACKGROUND

The application relates generally to use of NK-1 receptor antagonists. More particularly, the application relates the use of the NK-1 antagonist, tradipitant, for treatment of gastroparesis.

Gastroparesis is a serious medical condition characterized by delayed gastric emptying associated with the symptoms of nausea, vomiting, bloating, fullness after meals and abdominal pain, along with significant impairment of social and occupational functioning. The estimated prevalence of gastroparesis in the U.S. is over 5 million patients, many of whom remain undiagnosed. Gastroparesis affects women more frequently than men, and it can be of diabetic, idiopathic or other etiology. The only U.S. Food and Drug Administration approved treatment for gastroparesis is metoclopramide, approved in 1979, carries a black box warning and limitations of use of no more than 3 months due to its potential for severe side effects. Patients are therefore faced with limited therapeutic options, Clinical guidelines recommend, in addition to metoclopramide, the off label use of different drugs including erythromycin, domperidone (not approved in the U.S.), botulinum toxin injections, gastric stimulators and a variety of surgical procedures in an effort to achieve even temporary relief of some of the symptoms of the disease. Gastroparesis treatment represents a significant unmet medical need, as underscored by the testimonies of interested parties and advocacy organizations including the International Foundation for Gastrointestinal Disorders (IFFGD) and Gastroparesis Patient Association for Cures and Treatments, Inc. (G-Pact).

The precise underlying mechanisms leading to gastroparesis are currently poorly understood and are believed to be diverse in nature. The consensus suggests that gastroparesis arises from a dysregulation of the neuromuscular control of gastric movements that result in the timely emptying of stomach contents. The two key stimulatory neurotransmitters of the digestive system are acetylcholine and the neuropeptide substance P. Substance P acts by binding the neurokinin 1 receptor (NK-1R) at the gastric neuromuscular junction and it is believed that there is a functional interplay between the acetylcholine and NK-1R systems. Moreover, gastroparesis symptoms are also associated with aberrant physiology of the vagus nerve, which constitutes the major connection between the stomach and the central nervous system. Blockade of the NK-1Rs may have a dual and potentially therapeutic effect in gastroparesis by affecting gastric motility through a local action as well as affecting nausea and vomiting via a direct effect in the brain regions responsible for nausea and vomiting.

Tradipitant is a highly potent, selective, centrally penetrating, and orally active neurokinin-1 (NK-1) receptor antagonist, depicted below as the compound of Formula I

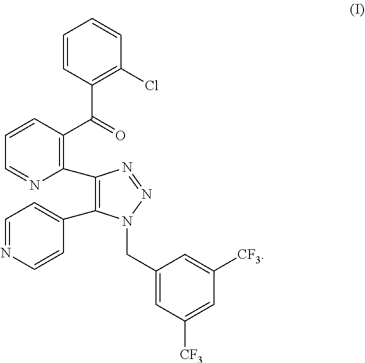 [SMILES: O=C(c1ccccc1Cl)c1cccnc1-c1nnn(Cc2cc(C(F)(F)F)cc(C(F)(F)F)c2)c1-c1ccncc1]

Tradipitant is disclosed in U.S. Pat. No. 7,320,994, and contains six main structural components: the 3,5-bis-trifluoromethylphenyl moiety, two pyridine rings, the triazol ring, the chlorophenyl ring, and the methanone. Tradipitant is known by the chemical names, 2-[1-[[3,5-bis(trifluoromethyl)phenyl]methyl]-5-(4-pyridinyl)-1H-1,2,3-triazol-4-yl]-3-pyridinyl](2-chlorophenyl)-methanone, and {2-[1-(3,5-bistrifluoromethylbenzyl)-5-pyridin-4-yl-1H-[1,2,3]triazol-4-yl]-pyridin-3-yl}-(2-chlorophenyl)-methanone, and has also been known as LY686017 and as VLY-686. U.S. Pat. No. 7,320,994 describes methods for using compounds, such as tradipitant, for treating a condition associated with an excess of tachykinins, most particularly where the condition associated with an excess of tachykinins is depression and anxiety. U.S. Pat. No. 7,320,994 further describes the possibility of using compounds, such as tradipitant, in other such diseases, i.e., because these compounds inhibit the physiological effects associated with an excess of tachykinins. The patent describes the usefulness of such compounds in the treatment of numerous other disorders related to tachykinin receptor activation including psychosis, schizophrenia, and other psychotic disorders; neurodegenerative disorders such as dementia, including senile dementia of the Alzheimer's type, Alzheimer's disease, AIDS-associated dementia, and Down Syndrome; demyelinating diseases such as multiple sclerosis and amyotrophic lateral sclerosis; and other neuropathological disorders, such as peripheral neuropathy, diabetic and chemotherapy-induced neuropathy, and post-herpetic and other neuralgias; acute and chronic obstructive airway diseases such as adult respiratory distress syndrome, bronchopneumonia, bronchospasm, chronic bronchitis, drivercough, and asthma; inflammatory diseases such as inflammatory bowel disease, psoriasis, fibrositis, osteoarthritis, and rheumatoid arthritis; disorders of the musculoskeletal system, such as osteoporosis; allergies such as eczema and rhinitis; hypersensitivity disorders such as poison ivy; ophthalmic diseases such as conjunctivitis, vernal conjunctivitis, and the like; cutaneous diseases such as contact dermatitis, atopic dermatitis, urticaria, and other eczematoid dermatites; addiction disorders such as alcoholism; stress-related somatic disorders; reflex sympathetic dystrophy such as shoulder/hand syndrome; dysthymic disorders; adverse immunological reactions such as rejection of transplanted tissues and disorders related to immune enhancement or suppression such as systemic lupus erythematosis; gastrointestinal disorders or diseases associated with the neuronal control of viscera such as ulcerative colitis, Crohn's disease, and irritable bowel syndrome; disorders of bladder function such as bladder detrusor hyper-reflexia and incontinence; atherosclerosis; fibrosin and collagen diseases such as scleroderma and eosinophilic fascioliasis; irritative symptoms of benign prostatic hypertrophy; disorders associated with blood pressure, such as hypertension; or disorders of blood flow caused by vasodilation and vasospastic diseases, such as angina, migraine, and Reynaud's disease; emesis, including chemotherapy-induced nausea and emesis; and pain or nociception, for example, that attributable to or associated with any of the foregoing conditions. Finally, the patent describes that such compounds are effective in amounts to be determined that range from 0.001 mg/kg/day to 100 mg/kg/day.

Tradipitant is known to be therapeutically administered through a variety of routes of administration by which it is bioavailable. U.S. Pat. No. 7,320,994 discloses administration of tradipitant by oral and parenteral routes, e.g., orally, by inhalation, subcutaneously, intramuscularly, intravenously, transdermally, intranasally, rectally, occularly, topically, sublingually, and buccally, with oral administration being generally preferred for treatment.

Crystalline Forms IV and V of tradipitant are disclosed in U.S. Pat. No. 7,381,826, and a process for preparing crystalline {2-[1-(3,5-bistrifluoromethylbenzyl)-5-pyridin-4-yl-1H-[1,2,3]triazol-4-yl]-pyridin-3-yl}-(2-chlorophenyl)-methanone, Form IV is disclosed in U.S. Pat. Nos. 8,772,496 and 9,708,291.

Additionally, methods of treatment of gastroparesis and gastric motility disorders are disclosed in International Patent Application Publication No. WO 2019/099883.

### BRIEF DESCRIPTION OF THE INVENTION

A first aspect of the invention provides an improved method for treating a patient diagnosed with gastroparesis by administering to said patient tradipitant in an amount effective to reduce one or more symptoms of gastroparesis. In this aspect, the improvement comprises maintaining said treatment for a period of at least one week before making an assessment with respect to the efficacy of said treatment.

A second aspect of the invention provides an improved method for treating a patient suffering from nausea, vomiting, bloating, postprandial fullness, or abdominal pain by administering to said patient tradipitant at a dose of 170 mg/day. In this aspect, the improvement comprises maintaining said treatment for a period of at least one week before making an assessment with respect to the efficacy of said treatment.

A third aspect of the invention provides a method comprising: identifying a patient in need of treatment for gastroparesis; administering to said patient tradipitant at a dose of 170 mg/day for a period of not less than one (1) week; and if said patient demonstrates a clinical significant response to tradipitant therapy, continuing said treatment with tradipitant.

A fourth aspect of the invention provides a method comprising: identifying a patient in need of treatment for nausea, vomiting, bloating, postprandial fullness, or abdominal pain; administering to said patient tradipitant at a dose of 170 mg/day for a period of not less than one (1) week; and if said patient demonstrates a clinical significant response to tradipitant therapy, continuing said treatment with tradipitant.

These and other aspects, advantages and salient features of the invention will become apparent from the following detailed description, which, when taken in conjunction with the annexed figure(s) disclose embodiments of the invention.

### BRIEF DESCRIPTION OF THE DRAWINGS

FIG. 1 illustrates the effect of tradipitant on NK-1 agonist (GR73632, 3 pmol, icv)-induced foot-tapping behavior after oral administration.

FIG. 2 illustrates the effect of tradipitant on NK-1 agonist (GR73632, 3 pmol, icv)-induced foot-tapping behavior after oral administration: comparison with other NK-1 antagonists, aprepitant and CP-122721: dose response.

FIG. 3 illustrates the effect of tradipitant on NK-1 agonist (GR73632, 3 pmol, icv)-induced foot-tapping behavior after oral administration: comparison with other NK-1 antagonists, aprepitant and CP-122721: time course.

FIG. 4 illustrates the effect of tradipitant on GR73632-induced vocalization in guinea pigs across a concentration range of 0.05 to 10 mg/kg.

FIG. 5 illustrates the duration of activity, i.e. suppression of NK-1 agonist-induced vocalization in guinea pigs, following a 0.1 mg/kg dose of tradipitant.

FIG. 6 illustrates the dose-dependent effects of tradipitant, and the effects of various NK-1 antagonists in the guinea pig vocalization assay.

FIG. 7 is a bar chart illustrating the median daily nausea score from 64 patients during a 4 week screening period (“Screen”) and during an open label extension (“OLE”) of the study described in Example 4.

FIG. 8 is a bar chart illustrating the percentage of days that subjects in the study described in Example 4 indicate that they are nausea-free, i.e., have a nausea score of 0, during the screening (“Screen”) and open label extension (“OLE”) periods.

The figures are intended to depict only typical aspects of the disclosure, and therefore should not be considered as limiting the scope of the disclosure.

### DETAILED DESCRIPTION OF THE INVENTION

At least one embodiment of the present invention is described below in reference to its application in connection with the use of tradipitant for the treatment of gastroparesis. Although some embodiments of the invention are illustrated relative to a specific disorder, i.e., gastroparesis, it is understood that the teachings are equally applicable to symptoms associated with gastroparesis, which may include nausea, vomiting, early satiety, postprandial fullness, dyspepsia (indigestion), functional dyspepsia, bloating, and abdominal pain.

In patients suffering from gastroparesis, patients can be treated by orally administering tradipitant in amounts and at a dosing frequency required to achieve plasma concentrations of at least about 100 ng/mL, e.g., 125 ng/mL or greater, 150 ng/mL or greater, 175 ng/mL or greater, 200 ng/mL or greater, or 225 ng/mL or greater. Such plasma concentration levels can be achieved, e.g., by orally administering to the patient tradipitant in a solid immediate release form comprising one or more pharmaceutically acceptable excipients and tradipitant in an amount of, e.g., 100 to 400 mg/day, 100 to 300 mg/day, 100 to 200 mg/day, or about 85-170 mg/day, which may be administered as, e.g., 50 to 200 mg bid, 50 to 150 mg bid, 50 to 100 mg bid, or about 85 mg bid as described in PCT publication WO 2016/141341 A1. With regard to dosing, “qd” refers to dosing once per day; “bid” dosing typically means dosing once in the morning and once in the evening, generally no less than about 8 hours or more than about 16 hours apart, e.g., 10 to 14 hours or 12 hours (Q12H).

As used herein, the terms “patient,” “subject,” and “individual” refer to human beings, as well as companion animals (e.g., dogs and cats) and other domesticated animals (e.g., horses, cattle, and sheep). It will be understood that the most preferred patient is a human being.

The invention further relates to the treatment of gastroparesis either prophylactically or therapeutically. Further, the terms “treatment” and “treating” are intended to refer to all processes wherein there may be a slowing, interrupting, arresting, controlling, or stopping of the progression of the disorders described herein, and is intended to include prophylactic treatment of such disorders, but does not necessarily indicate a total elimination of all disorder symptoms.

In one embodiment according to the invention, an improvement is provided for a method for treating a patient diagnosed with gastroparesis by administering to said patient tradipitant in an amount effective to reduce one or more symptoms of gastroparesis. Each of the one or more symptoms of gastroparesis are selected from the group consisting of: nausea, vomiting, bloating, postprandial fullness, and abdominal pain. In this embodiment, the amount effective to reduce (e.g., the severity, frequency, or intensity of) one or more symptoms of gastroparesis is 170 mg/day, e.g., 85 mg bid. In other embodiments, the dose may be provided in a dosage and formulation that provides exposure and/or plasma concentrations in the subject equivalent to those provided by, e.g., 85 mg bid in immediate release oral dosage forms. According to the present embodiment, said treatment is maintained for a period of at least one week before making an assessment with respect to the efficacy of said treatment. That is to say, an assessment regarding the efficacy of treatment is not made until at least one week after commencement of treatment. According to the present embodiment, the period of at least one week may be about one week, about two weeks, greater than two weeks, about two to about four weeks, two to four weeks, or longer. Such timing of the assessment of treatment efficacy provides improved accuracy with respect to the assessment, as treatment effect is observed at treatment week 1, and continues to increase through week 4.

According to another embodiment of the invention, an improvement is provided for a method for treating a patient suffering from nausea, vomiting, bloating, postprandial fullness, or abdominal pain by administering to said patient tradipitant in an effective amount. As used herein, the term “effective amount” refers to the dose of tradipitant administered to a patient that is effective in treating said disease, condition, or symptom. The effective amount may be, e.g., 170 mg/day, or more particularly 85 mg bid. The effective amount may further be provided in a dosage and formulation that provides exposure and/or plasma concentrations in the subject equivalent to those provided by, e.g., 85 mg bid in immediate release oral dosage forms. According to the present embodiment, said treatment is maintained for a period of at least one week before making an assessment with respect to the efficacy of said treatment. That is to say, an assessment regarding the efficacy of treatment is not made until at least one week after commencement of treatment. According to the present embodiment, the period of at least one week may be about one week, about two weeks, greater than two weeks, about two to about four weeks, two to four weeks, or longer. Such timing of the assessment of treatment efficacy provides improved accuracy with respect to the assessment, as treatment effect is observed at treatment week 1, and continues to increase through week 4.

According to a further embodiment of the invention, a method is provided comprising the steps of: identifying a patient in need of treatment for gastroparesis; administering to said patient tradipitant at a dose of 170 mg/day for a period of not less than one (1) week; and if said patient demonstrates a clinical significant response to tradipitant therapy, continuing said treatment with tradipitant. The dose of 170 mg/day may particularly be 85 mg bid. In other embodiments, dose may be provided in a dosage and formulation that provides exposure and/or plasma concentrations in the subject equivalent to those provided by, e.g., 85 mg bid in immediate release oral dosage forms. According to the present embodiment, said treatment is maintained for a period of not less than one week before making an assessment with respect to the efficacy of said treatment. That is to say, an assessment regarding the efficacy of treatment is not made until at least one week after commencement of treatment. According to the present embodiment, the period of not less than one week may be about one week, about two weeks, greater than two weeks, about two to about four weeks, two to four weeks, or longer. Such timing of the assessment of treatment efficacy provides improved accuracy with respect to the assessment, as treatment effect is observed at treatment week 1, and continues to increase through week 4.

According to another embodiment of the present invention, a method is provided comprising the steps of: identifying a patient in need of treatment for nausea, vomiting, bloating, postprandial fullness, or abdominal pain; administering to said patient tradipitant at a dose of 170 mg/day for a period of not less than 1 weeks; and if said patient demonstrates a clinically significant response to tradipitant therapy, continuing said treatment with tradipitant. The dose of 170 mg/day may particularly be 85 mg bid. In other embodiments, the dose may be provided in a dosage and formulation that provides exposure and/or plasma concentrations in the subject equivalent to those provided by, e.g., 85 mg bid in immediate release oral dosage forms. According to the present embodiment, said treatment is maintained for a period of not less than one week before making an assessment with respect to the efficacy of said treatment. That is to say, an assessment regarding the efficacy of treatment is not made until at least one week after commencement of treatment. According to the present embodiment, the period of not less than one week may be about one week, about two weeks, greater than two weeks, about two to about four weeks, two to four weeks, or longer. Such timing of the assessment of treatment efficacy provides improved accuracy with respect to the assessment, as treatment effect is observed at treatment week 1, and continues to increase through week 4.

The skilled artisan will appreciate that additional preferred embodiments may be selected by combining the preferred embodiments above, or by reference to the examples given herein.

### EXAMPLES

### Examples 1-3: Pre-Clinical Studies

Tradipitant is a selective neurokinin-1 (NK-1) receptor antagonist. In vitro, tradipitant potently inhibits NK-1 receptor binding and antagonizes the effects of an NK-1 agonist in a functional assay. No significant activity is observed in a panel of 74 additional receptors, enzymes, and ion channels including the NK-2 and NK-3 receptors. By three different measures, tradipitant is also a potent centrally active NK-1 antagonist in vivo.

### Example 1: Mechanism Studies

Tradipitant inhibits [¹²⁵I]substance P (SP) binding to the NK-1 receptor expressed by IM-9 cells with a Kᵢ of 0.062 nM and inhibits the SP-induced mobilization of intracellular calcium in U373 cells with a K_{b} of 0.095 nM (Table 1).

TABLE 1
Affinity of tradipitant for NK-1 Receptors In Vitro

| | IM-9 Human | |
|---|---|---|
| | Cell Membrane | Calcium Mobilization |
| | Binding Kᵢ | in U373 Cells K_{b} |
| Antagonist | (nM) | (nM) |
| Tradipitant | 0.062 ± 0.012 | 0.095 ± 0.025 |
| Aprepitant | 0.14 ± 0.03 | 0.14 ± 0.01 |
| CP-122721 | 0.027 ± 0.01 | 0.034 ± 0.009 |

These potencies are similar to those observed with the NK-1 antagonists aprepitant (MK-869) and CP-122721. In addition, results from tradipitant evaluation in a panel of 74 other receptors, enzymes, and ion channels indicate that, at a test concentration of 1 μM, tradipitant does not exhibit any inhibition of binding greater than 50%. At the NK-2 and NK-3 receptors, the compound produces no significant inhibition. Therefore, tradipitant is a highly selective NK-1 antagonist in vitro.

As shown in Table 2, several of the major metabolites of tradipitant have an affinity for the NK-1 receptor based on a binding assay. These metabolites have high affinity for the NK-1 receptor.

TABLE 2
Affinity of tradipitant metabolites for NK-1
Receptors in vitro

| | | IM-9 Human |
|---|---|---|
| | | Cell Membrane |
| | | Binding Kᵢ |
| Metabolite | Designation | (nM) |
| LSN2081070 | M2 (Racemic) | 0.09 (n = 1) |
| LSN2107355 | M2 (S-enantiomer) | 0.08 (n = 1) |
| LSN2107357 | M2 (R-enantiomer) | 0.94 (n = 1) |
| LSN2195411 | M3 | 0.03 (n = 1) |
| LSN2195413 | M4 (Racemic) | 0.08 (n = 1) |

### Example 2: Efficacy Models for In Vivo Evaluation of Brain NK-1 Receptor

Occupancy and Efficacy of Tradipitant

### Example 2.1: Effects of Tradipitant on Centrally Administered NK-1

Agonist-Induced Foot-Tapping Behavior in Gerbils

Introduction

Differences in species selectivity of NK-1 receptors pose challenges to characterization of NK-1 receptor antagonists in vivo. Gerbil NK-1 receptors have previously been shown to be similar to those in humans Gerbils exhibit a characteristic stereotypic foot-tapping behavior in response to distress, fear, or aversive stimuli. Intracerebroventricular (icv) administration of substance P or a selective NK-1 receptor agonist such as GR73632 produces rapid rhythmic tapping of the hind feet lasting approximately 5 minutes, which can be inhibited by systemic administration of a brain penetrating antagonist of the NK-1 receptor. This response is selective for NK-1 agonists, since selective NK-2 and NK-3 agonists do not elicit a similar response. This behavioral response is further characterized and modified to enable identification and optimization, in vivo, of potent NK-1 receptor antagonists including tradipitant.

Methods

Male Mongolian gerbils (Harlan Sprague Dawley, Indianapolis, Ind.) weighing 26 to 40 grams are administered the selective neurokinin-1 receptor agonist GR73632 (3 μmol) via direct, vertical, free-hand intracerebroventricular (icv) injection to a depth of 4.5 mm below bregma with a cuffed 27-gauge needle attached to a 50 μl Hamilton syringe Immediately after injection, animals are placed individually into isolated chambers with pressure-sensitive velocimeter platform floors (San Diego Instruments acoustic startle apparatus) that detect and quantify vibration. The San Diego Instruments “SR” DOS-based computer program is used on a PC to record the number of foot-taps over the following 6 to 10 minutes, beginning 30 seconds after the floor is lightly tapped. Raw data are converted with a Microsoft® Excel® (Microsoft® and Excel® are registered trademarks of Microsoft Corp., Redmond, Wash.) macro that determines the number of events over threshold (125) in each 250-millisecond time bin over the 5.5 minutes following onset of observation. The total number and average intensity of events over the duration is determined. Total number of taps is analyzed with one-way ANOVA and post-hoc Dunnett's test using JMP statistical software.

A dose-response curve (with doses of 0.3, 1, 3 and 10 pmols, icv) is initially generated with the NK-1 agonist GR73632. Maximal foot-tapping behavior is achieved with 3 and 10 pmol doses; the 3 pmol dose is subsequently chosen as the dose of choice for antagonism experiments.

NK-1 antagonists are tested for their ability to attenuate GR73632-induced foot tapping. NK-1 antagonists are administered (po) via oral feeding tube at doses and time points specified in each experiment. All animals receive only one dose of NK-1 antagonists in all tests.

ED₅₀ Determinations/Dose-Response Tests

NK-1 antagonists are administered at multiple doses (at least 3; one dose per animal) and response to GR73632 is measured.

Duration of Action Tests

NK-1 receptor antagonists are administered at multiple pre-treatment times (one administration per animal) including at 0.5, 1, 2, 4, 7, 16, and 24 hours prior to GR73632 injection. GR73632 (Peninsula Labs, CA) is dissolved in saline. Tradipitant is dissolved in 1% CMC/0.5% SLS/0.085% PVP vehicle. CP-122721 and aprepitant are synthesized at Lilly Laboratories and dissolved in 10% ethanol/emulphor and 1% CMC/0.5% SLS/0.085% PVP respectively.

Results

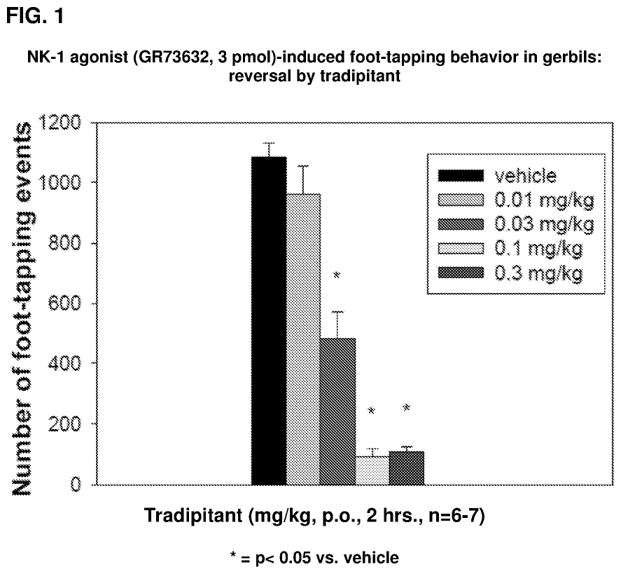

As shown in FIG. 1, orally administered tradipitant potently inhibits NK-1 agonist-induced foot-tapping behavior in gerbils 2 hours after administration of drug in a dose-dependent manner, with an ED₅₀ of 0.03±0.004 mg/kg (*p<0.05 compared to vehicle for 0.1 mg/kg and 0.3 mg/kg doses). Data shown in FIG. 1 are expressed in number of foot-tapping events occurring in five (5) minutes.

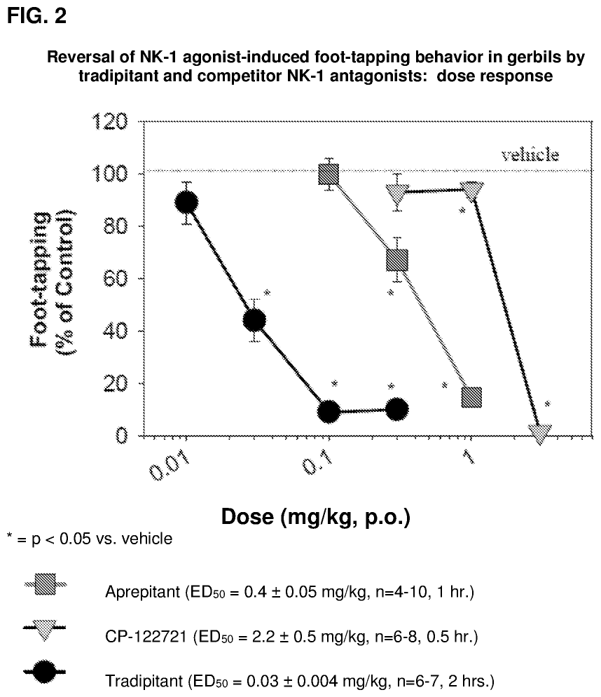

FIG. 2 depicts a comparison of the efficacy of tradipitant to that of other NK-1 antagonists, with data expressed as percent control of vehicle (vehicle response to 3 pmol GR73632). Tradipitant is found to be more potent than aprepitant (Merck, ED₅₀=0.42 mg/kg±0.05 mg/kg) and CP-122721 (Pfizer, ED₅₀=2.2 mg/kg±0.5 mg/kg).

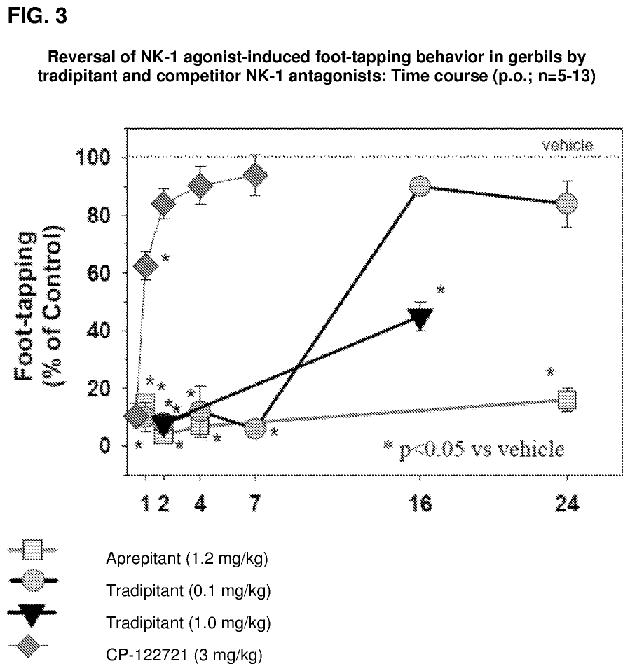

FIG. 3 depicts the effects of tradipitant over a time course on NK-1 agonist (GR 73632, 3 pmol, icv)-induced foot-tapping behavior after oral administration, compared with that of NK-1 antagonists aprepitant and CP-122721. Tradipitant (0.1 mg/kg, po) is found to significantly inhibit foot-tapping behavior up to 7 hours after administration but the effect is significantly diminished by 16 hours after administration at this dose. However, at a higher dose of 1 mg/kg, tradipitant shows greater than 50% inhibition of foot-tapping behavior 16 hours after administration. The duration of effect of tradipitant is longer than that of CP-122721 (up to 2 hours after administration, 3 mg/kg) while aprepitant (1 mg/kg) shows inhibition of foot-tapping behavior up to 24 hours after administration. Data are expressed as percent control of vehicle (vehicle response to 3 pmol GR73632).

Discussion

The effect of tradipitant on NK-1 agonist-induced foot-tapping behavior in gerbils suggests that tradipitant is a very potent, centrally acting NK-1 receptor antagonist in vivo in the gerbil with a relatively long duration of action.

### Example 2.2: Emetic Challenge Study in Beagle Dogs with Tradipitant

Introduction

Five male dogs are administered a single oral dose of 3 mg/kg aprepitant (a positive control), or tradipitant at 0.3, 1.0, and 3.0 mg/kg in a Latin-square design. An intravenous injection of 0.1 mg/kg apomorphine, a known emetic, is given alone, or 2 hours after administration of tradipitant or aprepitant. Each animal is administered a different dose on a particular dosing day, so that each dose of tradipitant, aprepitant, and apomorphine alone is represented. Over the five (5) weeks of the study, each animal receives each of the treatments, but only one per week. The purpose of this study is to determine if tradipitant suppresses apomorphine-induced emesis.

The low dose of tradipitant is 10 times the ED₅₀ in the gerbil foot-tapping model of NK-1 receptor antagonism (Example 1.2.1). The high dose is 100 times this efficacious dose, and is also the dose of aprepitant that has previously been determined to be efficacious against apomorphine-induced emesis in the dog. The mid dose of tradipitant is the approximate half-log interval between the low and high doses.

The oral route of administration is selected for tradipitant because this is the route proposed or currently used clinically. The intravenous route is typically used for experimental apomorphine administration. The beagle dog is considered an effective species for demonstration of antagonism of apomorphine-induced emesis.

Methods

A single oral dose of 0, 0.3, 1.0, or 3.0 mg/kg tradipitant, or 3.0 mg/kg aprepitant is administered to each male dog once a week in gelatin capsules. All animals are dosed over a period of five (5) weeks, with each dog receiving one of five different treatments on each day of dosing. A dose of 0.1 mg/kg apomorphine is administered by intravenous injection approximately two (2) hours after each administration of tradipitant or aprepitant. In cases where apomorphine is administered alone, without prior treatment with tradipitant or aprepitant, apomorphine is given at approximately the same time as when given in combination with tradipitant or aprepitant.

All dogs are fasted overnight prior to each treatment day and then fed approximately one (1) hour after oral dosing (approximately one (1) hour prior to administration of apomorphine). Individual doses are adjusted weekly for changes in body weight.

The dose regimen consists of a 5×5 Latin square design, in which each subject receives 1 dose or dose combination per week (6 day washout) as shown in Table 3 below.

TABLE 3
Latin Square Design

| | Study | Study | Study | Study | Study |
|---|---|---|---|---|---|
| | week 1 | week 2 | week 3 | week 4 | week 5 |
| Dog 1 | APO + 0.3 | APO + | APO + 3 | APO | APO + 1 |
| | mg/kg | aprepitant | mg/kg | | mg/kg |
| | tradipitant | | tradipitant | | tradipitant |
| Dog 2 | APO + | APO + 1 | APO | APO + 0.3 | APO + 3 |
| | aprepitant | mg/kg | | mg/kg | mg/kg |
| | | tradipitant | | tradipitant | tradipitant |
| Dog 3 | APO + 3 | APO | APO + | APO + 1 | APO + 0.3 |
| | mg/kg | | aprepitant | mg/kg | mg/kg |
| | tradipitant | | | tradipitant | tradipitant |
| Dog 4 | APO | APO + 0.3 | APO + 1 | APO + 3 | APO + |
| | | mg/kg | mg/kg | mg/kg | aprepitant |
| | | tradipitant | tradipitant | tradipitant | |
| Dog 5 | APO + 1 | APO + 3 | APO + 0.3 | APO + | APO |
| | mg/kg | mg/kg | mg/kg | aprepitant | |
| | tradipitant | tradipitant | tradipitant | | |

The number of emetic episodes is recorded for approximately one hour following the injection of apomorphine, and plasma concentrations at anticipated Tmax of tradipitant (2 hours post-dosing) are evaluated.

Results

Table 4 provides individual and mean and standard deviation values for the 2 hour plasma concentrations of tradipitant. All animals administered tradipitant have measurable levels at 2 hours post-dose. In general, plasma concentrations at 2 hours post-dose increase with increasing dose in a sub-proportional manner. As observed in other studies in dogs, the exposure to tradipitant is variable between animals. Individual animal data reveal no relationship between plasma concentrations and week of administration.

TABLE 4
Plasma concentrations of tradipitant (ng/ml)

| | 0.3 mg/kg | 1.0 mg/kg | 3.0 mg/kg |
|---|---|---|---|
| | Concentration | Concentration | Concentration |
| Administered | of tradipitant | of tradipitant | of tradipitant |
| Dose | (ng/ml) | (ng/ml) | (ng/ml) |
| Dog 1 | 51.20 | 175.58 | 122.73 |
| Dog 2 | 41.33 | 86.49 | 256.58 |
| Dog 3 | 90.93 | 240.84 | 316.20 |
| Dog 4 | 83.38 | 100.97 | 682.91 |
| Dog 5 | 22.59 | 61.56 | 119.79 |
| Mean (SD) | 57.89 (28.75) | 133.09 (73.71) | 299.64 (230.58) |

As shown in Table 5, emesis occurs after each treatment, with the largest incidence of emesis occurring in the apomorphine alone group. One dog (Dog 3) has a single episode of emesis at each dose of tradipitant and aprepitant; this dog also has the greatest number of emetic episodes with apomorphine alone (12). No emesis occurs in the remaining four (4) dogs at any dose of tradipitant or aprepitant. These dogs have an average of four (4) emetic episodes with apomorphine alone. The antiemetic effect of aprepitant supports the validity of this model.

TABLE 5
Emetic episodes by treatment group

| | | Total No. |
|---|---|---|
| | Dose level | Emetic |
| Test article* | (mg/kg) | Episodes |
| APO (control) | 0 | 28 |
| aprepitant | 3.0 | 1** |
| tradipitant | 0.3 | 1** |
| tradipitant | 1.0 | 1** |
| tradipitant | 3.0 | 1** |

*Apomorphine is administered as a challenge dose to all groups.
**All episodes occur in same dog (Dog 3).

Results of this study indicate that tradipitant is effective against apomorphine-induced emesis at each dose tested (0.3, 1.0, and 3.0 mg/kg).

### Example 2.3: Tradipitant Inhibits Substance P-Induced Vocalization in Guinea Pigs

Introduction

When introduced into the brain, the NK-1 receptor agonist substance P (SP) elicits distress vocalizations in the guinea pig that can be inhibited by NK-1 antagonists. This behavioral assay is used to demonstrate potency and CNS penetration of NK-1 antagonists in the guinea pig, a species that has receptor affinity for NK-1 antagonists that is similar to humans.

Methods

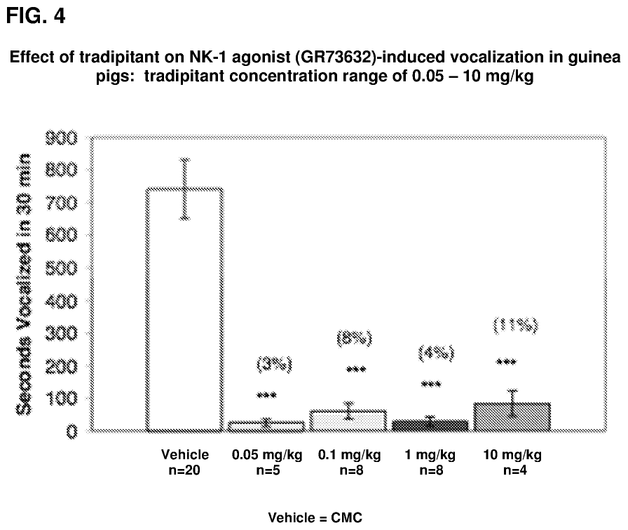

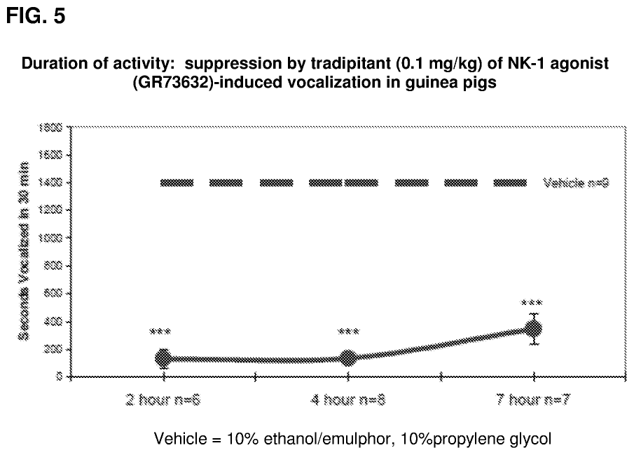

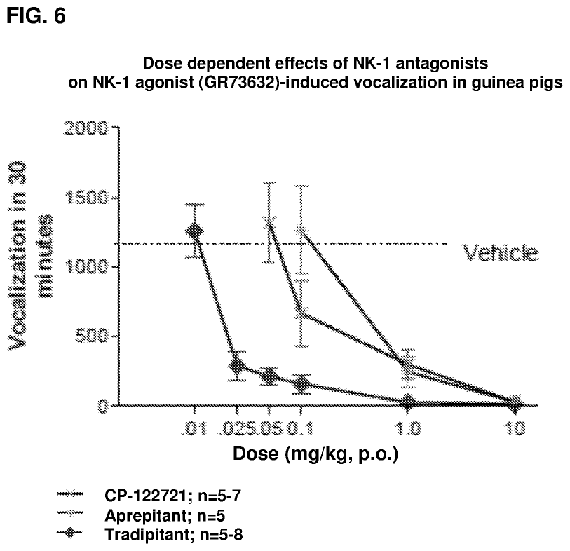

Male Dunkin/Hartley guinea pigs (200 to 250 grams) are orally administered either vehicle or an NK-1 antagonist. Approximately 45 minutes later (for dose response studies), the animals are anesthetized and 0.1 nmol of GR73632 (SP analog) in a vehicle volume of 5 μl is injected into the cerebral ventricle at the intersection of bregma and the midline of the skull. Animals are placed in a dark testing chamber located inside of a sound attenuation cubicle and vocalizations are recorded for 30 minutes following recovery from anesthesia. The time spent vocalizing is quantified for each animal. In the duration of action study, 0.1 mg/kg of tradipitant or vehicle solution is administered orally and then 2, 4, or 7 hours later, 0.1 nmol of GR73632 is administered into the cerebral ventricle as described above. Vocalizations are recorded and quantified as indicated above. Vehicle solutions are either CMC (FIG. 4 data) or an ethanol/emulphor solution (FIGS. 5 and 6). Data is analyzed using one-tailed t-tests.

Results

As shown in FIG. 4, oral administration of tradipitant produces significant inhibition of agonist-induced vocalization at doses of 10 mg/kg (p<0.001), 1.0 mg/kg (p<0.001), 0.1 mg/kg (p<0.001), and 0.05 mg/kg (p<0.001). Data shown parenthetically in FIG. 4 indicate percent of control response. Activity of tradipitant does not wane at the lower doses, indicating that even lower doses would be required to produce a dose response function.

As shown in FIG. 5, the effect of 0.1 mg/kg tradipitant is significantly active in suppressing agonist-induced vocalization at 7 hours following oral administration of the antagonist compound.

A second dose-response study compares potencies of tradipitant, aprepitant, and CP-122721. As shown in FIG. 6, all NK-1 antagonists tested produce significant inhibition of vocalization at 1 mg/kg. Only tradipitant retains significant inhibitory activity at and below 0.1 mg/kg. The minimum effective dose of tradipitant is found to be 0.025 mg/kg which produces highly significant (p<0.001) inhibition of vocalization compared to controls. (Vehicle was ethanol/emulphor; vehicle groups were n=5-14 per compound.)

Discussion

Tradipitant significantly inhibits NK-1 agonist-induced vocalization in guinea pigs, indicating that this compound is an orally available and brain-penetrant NK-1 antagonist. The minimum effective dose (MED) that produces this effect is 0.025 mg/kg. Tradipitant, administered orally, is shown to have a duration of activity that exceeds 7 hours. In this experimental paradigm, tradipitant is substantially more potent than aprepitant and CP-122721.

### Example 2.4: Occupancy of NK-1 Receptors

A tracer NK-1 antagonist compound (GR205171) is used to evaluate the ability of other NK-1 antagonists to occupy the brain NK-1 receptors. In these studies, the test compounds are administered orally and the tracer compound is administered intravenously afterward. The occupancy of the NK-1 receptors is evaluated by quantitating the amount of the tracer compound bound to the brain NK-1 receptors after increasing doses of the test compounds. Using this paradigm, tradipitant has an estimated ED₅₀ of 0.04 mg/kg p.o. and is substantially more potent than the other antagonists evaluated.

### Examples 3-5: Clinical Studies

### Example 3: Gastrointestinal Motility

A single-center, randomized, double-blind, placebo-controlled study is conducted to investigate the effect of tradipitant on small bowel transit time. A total of 15 healthy subjects, 12 men and 3 women, between the ages of 19 and 63 years are enrolled in the study and receive at least 1 dose of study medication. A total of 13 subjects complete the study. Subjects are randomized to receive 20 mg of tradipitant, 200 mg of tradipitant, or placebo as a single oral dose within each of 3 periods, co-administered with a capsule radiolabeled with a maximum of 1MBq ¹¹¹In. Four hours post-dose, all subjects also receive a second capsule radiolabeled with a maximum of 4MBq ^{99m}Tc. Each subject receives all 3 doses during the study. For all dosing regimens, in vivo gamma scintigraphic studies are performed at predetermined intervals, and the following scintigraphic parameters are analyzed: onset and completion of gastric emptying, onset and completion of colon arrival, initiation and completion of small bowel transit, and initial and complete disintegration of the capsule (anatomical location and time).

A statistically significant effect of tradipitant on small bowel transit time is observed in the study. No effect on gastric emptying is observed in this study. However, the study is underpowered with respect to this parameter.

### Example 4: Gastroparesis

In a phase II study of tradipitant in patients with gastroparesis, participants initially enter 4 week screening period prior to treatment with tradipitant, during which they answer a daily symptom questionnaire rating their nausea severity on a scale of 0 to 5, in which 0 represents no nausea, 1 represents very mild nausea, 2 represents mild nausea, 3 represents moderate nausea, 4 represents severe nausea, and 5 represents very severe nausea. After the 4 week screening period, patients enter the double blind randomized portion of the study, in which they receive either tradipitant 85 mg bid, or placebo. After 4 weeks of blinded study drug treatment, patients have the opportunity to enter an additional 8 week open label extension, during which each subject receives tradipitant 85 mg bid. 64 subjects participate in at least one week of the open label tradipitant dosing portion of the study. The average daily diarized nausea scores are reported for subjects during the 4 week screening period and during the up to 8 week open label extension.

Results

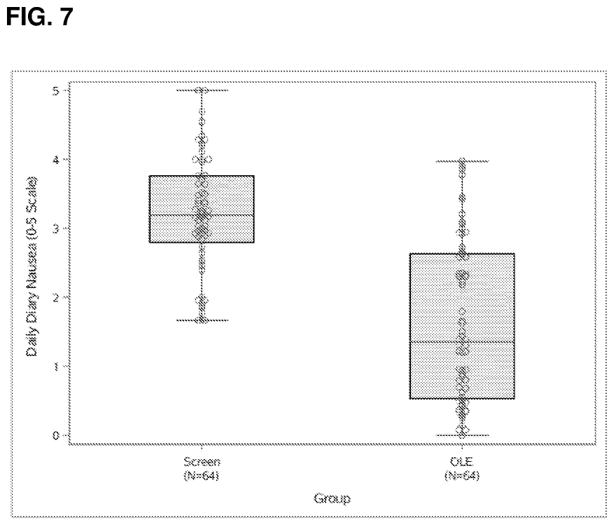

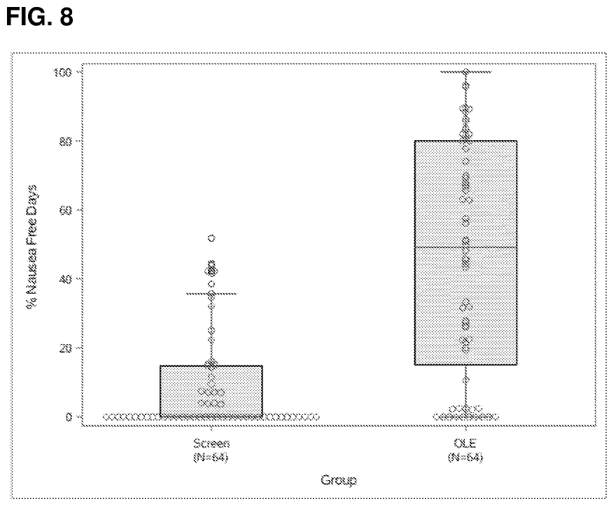

During the screening period, i.e. prior to randomization, subjects report a mean daily nausea score over the 4 week period of about 3.20 (±std. dev. of 0.83) on the 0 to 5 scale, i.e. greater than “moderate nausea.” During the open label portion of the study, during which the same 64 patients receive 85 mg bid tradipitant for a duration of 1 to 8 weeks, the same patients report a mean daily nausea score of about 1.63 (±std. dev. of 1.17) on the 0 to 5 scale. FIG. 7 illustrates the median daily nausea scores for the 64 subjects in the screening period (3.19) and open label extension period (1.35) respectively. Additionally, during the screening period prior to randomization, subjects report a mean 9.39% days (±std. dev. of 14.98) nausea-free, while during the open label extension, subjects report a mean 45.91% of days (±std. dev. of 33.64) nausea-free. FIG. 8 illustrates the median percentage of nausea-free days during the screening period (0%) and during the open label extension (49.11%).

Conclusions

The results described above and illustrated in FIGS. 7 and 8 demonstrate that in subjects suffering from gastroparesis, treatment with 85 mg bid tradipitant significantly improves both subjects' average daily nausea scores and experiences of nausea-free days. Compared to pre-treatment, administration of 85 mg bid tradipitant results in subjects reporting average daily nausea scores that are reduced by approximately one half, and average percentage of nausea-free days that are doubled or greater.

### Example 5: Gastroparesis

In a phase II study of tradipitant in patients with idiopathic and diabetic gastroparesis, participants enter a 4 week screening period prior to treatment with tradipitant, during which they answer a daily symptom questionnaire rating their nausea severity on a scale of 0 to 5, in which 0 represents no nausea, 1 represents very mild nausea, 2 represents mild nausea, 3 represents moderate nausea, 4 represents severe nausea, and 5 represents very severe nausea. After the 4 week screening period, 141 gastroparesis patients in an Intent to Treat (ITT) population enter the 4 week double blind randomized portion of the study, in which they receive either tradipitant 85 mg bid, or placebo. Several symptom severity scales are used to assess gastroparesis symptoms, including the Gastroparesis Symptom Index (GCSI), Patients Assessment of Upper Gastrointestinal Disorders-Symptoms (PAGI-SYM), and Patient Global Impression of Change (PGI-C), as well as a Clinician Global Impression of Severity (CGI-S). After 4 weeks of blinded study drug treatment, patients have the opportunity to enter an additional 8 week open label extension, during which each subject receives tradipitant 85 mg bid.

Results

During the double blind randomized portion of the study, patients receiving tradipitant 85 mg bid demonstrate a change of −1.2 in nausea score as measured by patient daily diaries, as compared to −0.7 for placebo (p=0.0099). Additionally, patients receiving tradipitant 85 mg bid report an increased number of nausea free days (addition of 28.8% of days) compared to placebo (addition of 15% for placebo) (p=0.0160).

Patients receiving tradipitant 85 mg bid also show significant improvement in secondary endpoints, including the several key scales reflecting overall gastroparesis symptoms: GCSI (p=0.0223); PAGI-SYM (p=0.0497); CGI-S (p=0.0207); PGI-C (p=0.0429). See Table 6.

TABLE 6
Study VLY-686-2301 Results Summary
ITT Population (n = 141)

| | Tradipitant | Placebo | |
|---|---|---|---|
| | n = 73 | n = 68 | p-value |
| Primary End Point | | | |
| DD-Nausea | −1.25 | −0.73 | 0.0099 |
| Secondary End Points | | | |
| DD-% Nausea Free Days | 28.8 | 15.0 | 0.0160 |
| GCSI | −0.93 | −0.58 | 0.0223 |
| PAGI-SYM | −0.93 | −0.65 | 0.0497 |
| CGI-S | −1.13 | −0.74 | 0.0207 |
| PGI-C | 2.66 | 3.06 | 0.0429 |

Vomiting Population (n = 101)

| | Tradipitant | Placebo | |
|---|---|---|---|
| | n = 58 | n = 43 | p-value |
| Primary End Point: | | | |
| DD-Nausea | −1.43 | −0.42 | <0.0001 |
| Secondary End Points: | | | |
| DD-% Nausea Free Days | 32.3 | 7.6 | 0.0003 |
| GCSI | −1.10 | −0.60 | 0.0078 |
| PAGI-SYM | −1.06 | −0.69 | 0.0294 |
| CGI-S | −1.24 | −0.79 | 0.0229 |
| PGI-C | 2.52 | 3.24 | 0.0018 |

Abbreviations used in Table 6 include: Daily Diary Nausea Score, 0-5 (DD-Nausea); Daily Diary Nausea Free Days, percent 0-100 (DD-% Nausea Free Days); Intent to Treat (ITT); Gastroparesis Cardinal Symptom Index (GCSI); Patient Assessment of Gastrointestinal Disorders-Symptoms (PAGI-SYM); Clinician Global Impression of Severity (CGI-S); Patient Global Impression of Change (PGI-C). For DD-Nausea, DD-% Nausea Free Days, GCSI, PAGI-SYM and CGI-S, the values shown are changes from baseline.

In a subgroup analysis of patients that experience both nausea and vomiting at baseline (n=101), tradipitant shows highly significant effects on the primary endpoint of change in nausea score (change of −1.4 for tradipitant versus −0.4 for placebo, p=0.00002) as well as the number of nausea free days (an addition of 32.3% for tradipitant versus 7.6% for placebo, p=0.0003). Improvements are also seen in most of the core gastroparesis symptoms including nausea, vomiting, bloating, and fullness after meals, consistent with an overall improvement and no associated worsening of any of the core symptoms.

Most effects are apparent by the second week of treatment, although improvements continue through the fourth and last week of treatment in the tradipitant group. Adverse events are similar in the tradipitant and placebo arms, which confirms prior studies' findings that tradipitant is well tolerated. As shown in Table 7, effect in the tradipitant treatment arm of the study is detectable at week 1, with effect size increasing in week 3 and again in week 4.

TABLE 7
Effect size by week, tradipitant treatment arm

| | Daily Diary Nausea | | Effect size | |
|---|---|---|---|---|
| | Weekly Average | | estimate | P value |
| Tradipitant | Week 1 | Week 2 | 0.31399293 | 0.0005 |
| Tradipitant | Week 1 | Week 3 | 0.555567644 | <.0001 |
| Tradipitant | Week 1 | Week 4 | 0.59204039 | <.0001 |
| Tradipitant | Week 2 | Week 3 | 0.241574713 | 0.0173 |
| Tradipitant | Week 2 | Week 4 | 0.27804746 | 0.0205 |
| Tradipitant | Week 3 | Week 4 | 0.036472747 | 0.6746 |

Conclusions

The results described above and in Tables 6-7 illustrate that the greatest effect on the key symptom of nausea occurs in week 4 of treatment. This benefit is progressive over time, such that a trial of tradipitant treatment in gastroparesis patients should last at least one week, and may preferably last at least two weeks, or two to four weeks, or at least two to four weeks, in order to assess efficacy in a given individual patient. Additionally, gastroparesis patients in the vomiting subgroup demonstrate particularly strong treatment effect, indicating that treatment with tradipitant is particularly useful in this subgroup.

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1.-7. (canceled)

8. In a method for treating a patient suffering from nausea, vomiting, bloating, postprandial fullness, or abdominal pain by administering to said patient tradipitant at a dose of 170 mg/day, the improvement comprising:
maintaining said treatment for a period of at least one week before making an assessment with respect to the efficacy of said treatment.

9. The improvement according to claim 8, wherein the dose of 170 mg/day is 85 mg bid.

10. The improvement according to claim 8, wherein the period of at least one week is two weeks.

11. The improvement according to claim 8, wherein the period of at least one week is greater than two weeks.

12. The improvement according to claim 8, wherein the period of at least one week is about two to about four weeks.

13. The improvement according to claim 8, wherein the period of at least one week is two to four weeks.

14. A method comprising:
identifying a patient in need of treatment for gastroparesis;
administering to said patient tradipitant at a dose of 170 mg/day for a period of not less than 1 week; and
if said patient demonstrates a clinical significant response to tradipitant therapy, continuing said treatment with tradipitant.

15. The method of claim 14, wherein the dose of 170 mg/day is 85 mg bid.

16. The method of claim 14, wherein the period of not less than 1 week is about two weeks.

17. The method of claim 14, wherein the period of not less than 1 week is greater than two weeks.

18. The method of claim 14, wherein the period of not less than 1 week is about two to about four weeks.

19. The method of claim 14, wherein the period of not less than 1 week is two to four weeks.

20. A method comprising:
identifying a patient in need of treatment for nausea, vomiting, bloating, postprandial fullness, or abdominal pain;
administering to said patient tradipitant at a dose of 170 mg/day for a period of not less than 1 week; and
if said patient demonstrates a clinical significant response to tradipitant therapy, continuing said treatment with tradipitant.

21. The method of claim 20, wherein the dose of 170 mg/day is 85 mg bid.

22. The method of claim 20, wherein the period of not less than 1 week is two about weeks.

23. The method of claim 20, wherein the period of not less than 1 week is greater than two weeks.

24. The method of claim 20, wherein the period of not less than 1 week is about two to about four weeks.

25. The method of claim 20, wherein the period of not less than 1 week is two to four weeks.

26. The improvement according to claim 8, wherein the patient has a diagnosis of gastroparesis.

27. The method according to claim 20, wherein the patient has a diagnosis of gastroparesis.
